(19) United States
(12) Patent Application Publication
(10) Pub. No.: US 2006/0099230 A1
(43) Pub. Date: May 11, 2006

(54) Novel formulations of eprosartan with enhanced bioavailability

(76) Inventor: Chin-Chih Chiang, West Covina, CA (US)

Correspondence Address: SHIHONG NICOLAOU, 4931 ARROYO LINDO AVE., SAN DIEGO, CA, 92117, US

(21) Appl. No.: 10/986,539

(22) Filed: Nov. 10, 2004

(51) Int. Cl. A61K 31/4178; A61K 9/00

(52) U.S. Cl. 424/400; 514/397

(57) ABSTRACT

The invention relates to the composition of a novel formulation of eprosartan with enhanced bioavailability. The formulation comprises of eprosartan or a salt, solvate, or hydrate thereof, a solubilizer, and an emulsifier. A process for manufacturing, and methods of using the formulation to block angiotensin II receptors and to treat hypertension, congestive heart failure and renal failure are also provided.

### FIELD OF THE INVENTION

The invention relates generally to the bioenhanced formulations of eprosartan. More specifically, the invention provides the compositions and methods of use for the oral formulations of eprosartan with increased bioavailability. The invention further provides methods of preparing the bioenhanced formulation and the process for manufacturing.

### BACKGROUND OF THE INVENTION

Angiotensin II, a potent vasoconstrictor, is the most important active hormone of the rennin-angiotensin system, and it makes up an important determination of the pathophysiology of hypertension. A number of potent antagonists of angiotensin II receptor for oral administration have been developed, including such as losartan, candesartan, irbersartan, valsartan, telmisartan, eprosartan, tasosartan and zolosartan. Eprosartan, ((E)-.alpha.-[2-n-butyl-1-[(4-carboxyphenyl)methyl]-1H-imidazol-5-yl]methylene-2-thiophenepropionic acid) presents high affinity for the angiotensin II ATI receptor and is the subject of U.S. Pat. No. 5,185,351 (the '351 patent). This patent discloses a process for making the anhydrous form of (E)-.alpha.-[2-n-butyl- 1-(4-carboxyphenyl)methyl]-1H-imidazol-5-yl]methylene-2-thiophenepropionic acid and its methanesulfonate salt (eprosartan mesylate). Additionally, the '351 patent discloses conventional techniques for formulating (E)-.alpha.-[2-n-butyl-1-[(4-carboxyphenyl)methyl]-1H-imidazol-5-yl]methylene-2-thiophenepropionic acid. This compound is claimed to have utility in blocking angiotensin II receptors and to be useful in the treatment of hypertension, congestive heart failure and renal failure.

Eprosartan is marketed as tablet formulation containing eprosartan mesylate equivalent to 400 mg or 600 mg eprosartan zwitterions. The formulation comprises croscarmellose sodium, hydroxypropyl methycellulose, iron oxide, iron oxide yellow, lactose monohydrate, magnesium stearate, microcrystalline cellulose, polyethylene glycol, polysorbate 80, pregelatinized starch and titanium dioxide as inactive ingredients. According to Physician's Desk Reference, absolute bioavailability following a 300 mg oral dose of eprosartan is approximately 13%. Eprosartan plasma concentrations peak at 1 to 2 hours after an oral dose in the fasted state. Administering eprosartan with food delays absorption. Plasma concentrations of eprosartan increase in a slightly less than dose-proportional manner over the 100 mg to 800 mg dose range. The terminal elimination half-life of eprosartan following oral administration is typically 5 to 9 hours. Eprosartan does not significantly accumulate with chronic use.

Eprosartan can also be used in combination with other drugs, including such as a diuretic agent, hydrochlorothiazide. Commercial products are Teveten HCT 600/12.5 and Teveten 600/25, comprising 600 mg of eprosartan and 12.5 or 25 mg of hydrochlorothiazide, respectively. Eprosartan mesylate functions as an antagonist of angiotensin II receptor. Hydrochlorothiazide affects the renal tubular mechanisms of electrolyte reabsorption, directly increasing excretion of sodium and chloride in approximately equivalent amount. Indirectly, the diuretic action of hydrochlorothiazide reduces plasma volume, resulting in consequent increases in plasma rennin activity, increases in aldosterone secretion, increases in urinary potassium loss, and decreases in serum potassium. The rennin-aldosterone link is mediated by angiotensin II, and the coadministration of an angiotensin II receptor antagonist tends to reverse the potassium loss associated with these diuretics.

Because the variable and mean absolute bioavailability of eprosartan is only 13%, doses as high as 800 mg per day may be required for an effective treatment of hypertension, congestive heart failure and renal failure. Additionally, since the commercial form of the drug is as its mesylate, which becomes dihydrated during the formulation process, high dose tablets (e.g., 600 mg tablets weigh 1,200 mg) may be difficult to swallow. Therefore, there is a need for a formulation that enhances the bioavailability of eprosartan.

### SUMMARY OF THE INVENTION

The present invention provides the composition and methods of use of a novel formulation to increase the oral bioavailabilities of eprosartan. The formulation comprises eprosartan and a solubilizer and/or an emulsifier.

In one embodiment, the formulation comprises eprosartan or a salt, solvate or hydrate thereof, a solubilizer and/or an emulsifier. The solubilizer is selected from water, propylene glycol, ethanol, labrasol, propylene glycol-400 (PEG-400) and lactic acid. The emulsifier is selected from tocopherols, tocotrienosl, Gelucire, and Capmul.

In another embodiment, the preparation of the formulation comprises the steps of mixing the active ingredient in the presence of a solubilizer and/or an emulsifier, optionally adding one or more pharmaceutical acceptable excipients; and filling the resulting liquid into a capsule. The liquid can be a suspension, an emulsion or a solution.

In another related aspect, the novel formulation of eprosartan is used for blocking angiotensin II receptors, treating hypertension, congestive heart failure, and renal failure.

The present invention also provides formulations comprising a second pharmaceutically active compound selected from the group consisting of a diuretic, a vasodilator, a centrally acting α-agonists, a postganglionic adrenergic neuron blocker, a α-adrenergic blocker, a calcium channel blocker, an β-adrenoceptor blocker, a renin inhibitor, and an angiotensin converting enzyme inhibitor.

### BRIEF DESCRIPTION OF THE DRAWINGS

FIG. 1 illustrates the plasma concentrations of eprosartan vs. time following the oral administration of the eprosartan formulations.

### DETAILED DESCRIPTION OF THE INVENTION

Eprosartan ((E)-.alpha.-[2-n-Butyl-1-[(4-carboxyphenyl)methyl]-1H-imidazol-5-yl]methylene-2-thiophenepropionic acid) and eprosartan mesylate are potent in blocking angiotensin II receptors, and can be used in the treatment of hypertension, congestive heart failure and renal failure. Eprosartan is disclosed in U.S. Pat. No. 5,185,351, and is herein incorporated by reference in its entirety.

Eprosartan salts refer to both acid and base addition salts. Pharmaceutical acceptable acid addition salts of eprosartan can be formed with appropriate organic or inorganic acids by methods known in the art. For example, the base is reacted with a suitable inorganic or organic acid in an aqueous miscible solvent such as ethanol with isolation of the salt by removing the solvent or in an aqueous immiscible solvent when the acid is soluble therein, such as ethyl ether or chloroform, with the desired salt separating directly or isolated by removing the solvent. Representative examples of suitable acids are maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids. Acceptable base addition salts of eprosartan can be prepared by known methods from organic and inorganic bases, including nontoxic alkali metal and alkaline earth bases, for example, calcium, lithium, sodium, and potassium hydroxide; ammonium hydroxide, and nontoxic organic bases, such as triethylamine, butylamine, piperazine, meglumine, choline, diethanolamine, and tromethamine.

The present invention provides the compositions of a novel formulation of eprosartan or a salt, solvate, or hydrate thereof, which exhibits increased bioavailability. The pharmaceutical formulation comprises eprosartan or a salt, solvate, or hydrate thereof, a solubilizer and/or an emulsifier. The use of tocopherols, tocotrienols and the derivatives thereof have been found to be useful in pharmaceutical formulations to enhance the bioavailability. Vitamin E TPGS is one familiar member of the tocopherol compounds, and it is the trade name of Eastman. The chemical name of Vitamin E TPGS is α-tocopherol polyethylene glycol 1000 succinate.

The solubilizer for eprosartan formulation is selected from propylene glycol, polyethylene glycols, derivatives of polyols, aliphatic alcohols and aliphatic esters. The emulsifier is selected from Vitamin E TPGS (α-tocopherol polyethylene glycol 1000 succinate), Gelucire 44/14 (a mixture of C8-18 fatty acids ethoxylated C8-18 glycerides), Cremophors (PEG-60 hydrogenated castor oil), Capmuls, Tweens (polyoxyethylene sorbitan esters), Spans (sorbitan fatty acid esters), Brij (polyoxyethylene alcohols), and Myrj (polyoxyethylene fatty acid esters).

Oral administration of eprosartan mesylate in a conventional formulation shows a low bioavailability of 13%. The low oral bioavailability of eprosartan is limited by the solubility, rather than the metabolism in cytochrome P450 in liver. The novel formulations as provided in the present invention enhance the oral absorption of eprosartan by increasing the solubility of eprosartan in the gastro-intestinal fluids.

A formulation of the present invention can also include other components such as a pharmaceutical acceptable excipient. The excipients can be, for example, co-solvent such as labrasol, ethanol, and Capmul PG-8 (a trade name of Abitec Corporation).

In the present invention, eprosartan is formulated as a suspension, an emulsion or a solution. The formulation comprises 5%-60% (w/w) eprosartan, 5%-95% (w/w) solubilizer, and 5%-95% of emulsifier. The preferred solubilizer is propylene glycol; and the preferred emulsifier is Vitamin E TPGS.

The general process of preparing the formulations comprises the steps of mixing the liquid ingredients first, and adding and mixing the active ingredients until homogeneous, and filling the liquid into capsules.

A dissolution profile (i.e., the percentage of eprosartan released into the solution versus time profile) of the novel formulation was studied and characterized as a fast dissolving profile. Consequently, the ingestion of the capsule provides a considerably high serum concentration of eprosartan rapidly. The dissolution study was conducted under US Pharmacopoeia XXIII, Dissolution method I, in a basket apparatus at temperature 37° C., in 0.01N HCl medium and at 50 r.p.m. The dissolution profile demonstrated essentially 100% of the eprosartan being released from the formulation within 60 minutes from the start of the dissolution test.

The dissolution samples thus obtained were assayed by a high pressure liquid chromatography (HPLC) method for eprosartan concentrations. HPLC conditions are described as the followings.
Column: C₁₈ Capcell Pak column of 4.6 mm×250 mm, 5 μm
Mobile phase: 24% acetonitrile, and 76% phosphate buffer (pH 3.0)
Wavelength of detection: 235 nm

Oral bioavailability can be assessed by measuring AUC or Cₘₐₓ, both parameters are well known in the art. AUC is a determination of the Area Under the Curve plotting the serum or plasma concentration of drug along the Y-axis against time along the X-axis. Cₘₐₓ is an abbreviation for the maximum drug concentration achieved in the serum or plasma of the test subject. An enhanced oral bioavailability of eprosartan as measured by AUC was shown in the formulations of the present invention. In a more preferred embodiment of the formulations, the bioavailability as measured by AUC, was increased by 162%. That is, to an absolute bioavailability of at least 34%.

Eprosartan may be co-administered with other pharmaceutically active compounds, for example, in physical combination or by sequential administration. The formulation of the present invention may include another active compound for convenience of administration. Thus, this invention also relates to pharmaceutical compositions comprising eprosartan, a salt, solvate or hydrate thereof, an solubilizer and/or an emulsifier, and a second pharmaceutically active compound selected from the group consisting of a diuretic, a vasodilator, a centrally acting α-agonists, a postganglionic adrenergic neuron blocker, a α-adrenergic blocker, a calcium channel blocker, an β-adrenoceptor blocker, a renin inhibitor, and an angiotensin converting enzyme inhibitor.

Examples of compounds which may be included in pharmaceutical compositions in combination with eprosartan are diuretics, particularly a thiazide diuretic, such as hydrochlorothiazide, or a loop diuretic, such as furosemide, or a potassium-sparing diuretic, such as amiloride, vasodilators, such as minoxidil, calcium channel blockers, particularly dihydropyridine antagonists, such as amlodipine, isradipine, felodipine, nicardipine, or nifedipine, β-adrenoceptor blockers, such as atenolol, betaxolol, bisoprolol, carteolol, or propranolol, renin inhibitors, such as enalkinen, and angiotensin converting enzyme inhibitors, such as captopril or enalapril. Preferably, the pharmaceutical composition contains 200-400 mg of eprosartan in combination with 6.25-25 mg of hydrochlorothiazide.

The following examples are provided to further explain the invention.

### EXAMPLE

### Example 1

The Compositions and Method of Preparation for Formulation 1

Formulation 1

| Ingredient | Weight % |
|---|---|
| Eprosartan mesylate | 10% |
| Propylene glycol | 20% |
| Water | 10% |
| Vitamin E TPGS | 60% |
| TOTAL | 100% |

Method for the Preparation of Formulation 1:

a. Mix propylene glycol and water in a suitable container.

b. Add Vitamin E TPGS in the same container, and mix until homogeneous. The temperature is maintained at 50° C. with constant stirring to obtain a homogeneous solution.

c. Add eprosartan mesylate into the same container, and mix with the other excipients into a homogeneous suspension.

d. Fill the suspension into hard gelatin capsules.

e. Seal the capsules and allow the formulation to cool down to the room temperature.

### Example 2

The Compositions and Method of Preparation for Formulation 2

Formulation 2

| Ingredient | Weight % |
|---|---|
| Eprosartan mesylate | 10% |
| Propylene glycol | 20% |
| Water | 10% |
| Ethanol | 10% |
| Labrasol | 10% |
| Vitamin E TPGS | 40% |
| TOTAL | 100% |

Method for the Preparation of Formulation 2:

a. Mix propylene glycol, water, ethanol and labrasol in a suitable container.

b. Add Vitamin E TPGS in the same container. The temperature is maintained at 50° C. with constant stirring to obtain a homogeneous solution.

c. Add eprosartan mesylate into the same container, and mix with the other excipients into a homogeneous suspension.

d. Fill the suspension into hard gelatin capsules.

e. Seal the capsules and allow the formulation to cool down to the room temperature.

### Example 3

The Compositions and Method of Preparation for Formulation 3

Formulation 3

| Ingredient | Weight % |
|---|---|
| Eprosartan mesylate | 12% |
| Propylene glycol | 20% |
| Water | 10% |
| Ethanol | 10% |
| Polyethylene glycol-400 | 20% |
| Vitamin E TPGS | 28% |
| TOTAL | 100% |

Method for the Preparation of Formulation 3:

a. Mix propylene glycol, water, ethanol and polyethylene glycol-400 in a suitable container.

b. Add Vitamin E TPGS in the same container. The temperature is maintained at 50° C. with constant stirring to obtain a homogeneous solution.

c. Add eprosartan mesylate into the same container, and mix with the other excipients into a homogeneous suspension.

d. Fill the suspension into hard gelatin capsules.

e. Seal the capsules and allow the formulation to cool down to the room temperature.

### Example 4

The Compositions and Method of Preparation for Formulation 4

Formulation 4

| Ingredient | Weight % |
|---|---|
| Eprosartan mesylate | 10% |
| Propylene glycol | 20% |
| Labrasol | 10% |
| Vitamin E TPGS | 60% |
| TOTAL | 100% |

Method for the Preparation of Formulation 4:

a. Mix propylene glycol and labrasol in a suitable container.

b. Add Vitamin E TPGS in the same container. The temperature is maintained at 50° C. with constant stirring to obtain a homogeneous solution.

c. Add eprosartan mesylate into the same container, and mix with the other excipients into a homogeneous suspension.

d. Fill the suspension into hard gelatin capsules.

e. Seal the capsules and allow the formulation to cool down to the room temperature.

### Example 5

The Compositions and Method of Preparation for Formulation 5

Formulation 5

| Ingredient | Weight % |
|---|---|
| Eprosartan mesylate | 20% |
| Propylene glycol | 8% |
| Vitamin E TPGS | 72% |
| TOTAL | 100% |

Method for the Preparation of Formulation 5:

a. Mix propylene glycol and Vitamin E TPGS in a suitable container. The temperature is maintained at 50° C. with constant stirring to obtain a homogeneous solution.

b. Add eprosartan mesylate into the same container, and mix with the other excipients into a homogeneous suspension.

c. Fill the suspension into hard gelatin capsules.

d. Seal the capsules and allow the formulation to cool down to the room temperature.

### Example 6

The Compositions and Method of Preparation for Formulation 6

Formulation 6

| Ingredient | Weight % |
|---|---|
| Eprosartan Mesylate | 20% |
| Gelucire 44/14 | 24% |
| Capmul PG-8 | 56% |
| TOTAL | 100% |

Method for the Preparation of Formulation 6:

a. Mix Capmul PG-8 and Gelucire 44/14 in a suitable container. The temperature is maintained at 50° C. with constant stirring to obtain a homogeneous solution.

b. Add eprosartan mesylate into the same container, and mix with the other excipients into a homogeneous suspension. Maintain the temperature at 50° C.

c. Fill the suspension into hard gelatin capsules.

d. Seal the capsules and allow the formulation to cool down to the room temperature.

### Example 7

The Compositions and Method of Preparation for Formulation 7

Formulation 7

| Ingredient | Weight % |
|---|---|
| Eprosartan mesylate | 10% |
| Lactic acid | 75% |
| Vitamin E TPGS | 15% |
| TOTAL | 100% |

Method for the Preparation of Formulation 7:

a. Mix lactic acid and Vitamin E TPGS in a suitable container. The temperature is maintained at 50° C. with constant stirring to obtain a homogeneous solution.

b. Add eprosartan mesylate into the same container, and mix with the other excipients into a homogeneous suspension.

c. Fill the suspension into hard gelatin capsules.

d. Seal the capsules and allow the formulation to cool down to the room temperature.

### Example 8

The Compositions and Method of Preparation for Formulation 8

Formulation 8

| Ingredient | Weight % |
|---|---|
| Eprosartan mesylate | 20% |
| Hydrochlorothiazide | 3.33% |
| Propylene Glycol | 8% |
| Vitamin E TPGS | 68.67% |
| TOTAL | 100% |

Method for the Preparation of Formulation 8:

a. Mix propylene glycol and Vitamin E TPGS in a suitable container. The temperature is maintained at 50° C. with constant stirring to obtain a homogeneous solution.

b. Add eprosartan mesylate into the same container, and mix with the other excipients into a homogeneous suspension.

c. Add hydrochlorothiazide into the same container, and further mix with the other ingredients into a homogeneous suspension.

d. Fill the suspension into hard gelatin capsules.

e. Seal the capsules and allow the formulation to cool down to the room temperature.

### Example 9

The Dissolution Study of Formulation 5

Dissolution study was conducted using a modified USP basket method. Dissolution medium was 900 mL of 0.1N HCl solution in a dissolution vessel. The temperature was maintained at 37° C. with circulation water.

The dissolution profile of Formulation 5 indicated that eprosartan mesylate is completely dissolved into the dissolution medium in 60 minutes.

### Example 10

The Pharmacokinetic Study of Selected Formulations

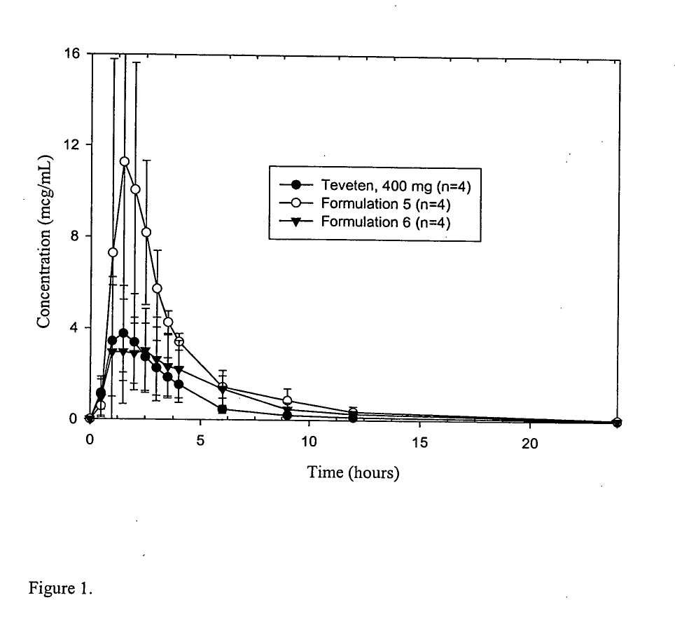

Formulations 5 and 6 were studied in human volunteers to evaluate the relative oral bioavailabilities. The studies were conducted using Formulations 5 and 6 with 100 mg eprosartan mesylate in each capsule, and using Teveten, 400 mg, a marketed tablet manufactured by Solvay as reference. It is a parallel single dose study. Four volunteers participated in each group. In the study group, each volunteer was administered with 2 eprosartan capsules of Formulation 5 or Formulation 6. In the reference group, each volunteer was administered with one Teveten tablet, 400 mg. Blood samples were withdrawn for quantitation of eprosartan. Normalized plasma concentration vs. time profiles of eprosartan are shown in FIG. 1. The pharmacokinetic data comparing Formulations 5 and 6 to Teveten are summarized in Table 1.

TABLE 1
Mean Values of Pharmacokinetic
Parameters and Relative Bioavailability

| | Teveten | Formulation 5 | Formulation 6 |
|---|---|---|---|
| Parameters | 400 mg × 1 | 100 mg × 2 | 100 mg × 2 |
| AUC_{0-∞} (ng/mL · hr) | 14343 ± 6823 | 15333 ± 5854 | 7475 ± 4703 |
| Cₘₐₓ (ng/mL) | 4084 ± 2112 | 5162 ± 3151 | 1514 ± 1099 |
| Tₘₐₓ (hr) | 1.25 ± 0.29 | 21.3 ± 0.75 | 1.75 ± 0.65 |
| *BA (%) | 100 | 262.2 | 127.3 |

*BA (%) = (AUC_{0-∞}/Dose)_{Capsule formulations}/(AUC_{0-∞}/Dose)_{Teveten} × (520.63/424.52)

Dose in Formulations 5 and 6 is based on the eprosartan mesylate salt with MW 520.63 and dose in Teveten formulation is based on its free base form with MW 424.52.

Pharmacokinetic data as represented in Table 1 indicated that Formulation 5 and Formulation 6 exhibit relative oral bioavailabilities of 262.2% and 127.3% as compared to Teveten, respectively.

Although the invention has been described with reference to the examples described herein, it will be understood that modifications and variations are encompassed within the spirit and scope of the invention.

What is claimed is:

1. A pharmaceutical formulation comprising eprosartan or a salt, solvate, or hydrate thereof, a solubilizer and an emulsifier.

2. The formulation as in claim 1, wherein the eprosartan or a salt, solvate or hydrate thereof, is eprosartan mesylate.

3. The formulation as in claim 1, wherein the solubilizer is selected from water, propylene glycol, ethanol, labrasol, polyethylene glycol-400, and lactic acid.

4. The formulation as in claim 3, wherein the solubilizer is propylene glycol.

5. The solubilizer as in claim 3, wherein water is in the range of 5-75% (w/w), propylene glycol is in the range of 8-40% (w/w), ethanol is in the range of 8-40% (w/w), labrasol is in the range of 5-80% (w/w), polyethylene glycol-400 is in the range of 20-80% (w/w), and lactic acid is in the range of 30-90% (w/w).

6. The formulation as in claim 1, wherein the emulsifier is selected from tocopherols, tocotrienols, Gelucire, and Capmul.

7. The emulsifier as in claim 6, wherein the tocopherol is Vitamin E TPGS.

8. The emulsifier as in claim 6, wherein tocopherol is in the range of 8-80% (w/w), tocotrienol is in the range of 8-80% (w/w), Gelucire 44/14 is in the range of 15-60% (w/w), and Capmul PG-8 is in the range of 20-70% (w/w).

9. A pharmaceutical formulation comprising the compounds according to claim 1, and a second pharmaceutically active compound selected from the group consisting of a diuretic, a vasodilator, a centrally acting α-agonists, a postganglionic adrenergic neuron blockers, a α-adrenergic blocker, a calcium channel blocker, an β-adrenoceptor blocker, a renin inhibitor, and an angiotensin converting enzyme inhibitor.

10. The formulation as in claim 9 wherein the second pharmaceutically active compound is a diuretic.

11. The formulation as in claim 10 wherein the diuretic is hydrochlorothiazide.

12. The formulation as in claim 9 wherein the second pharmaceutically active compound is a loop diuretic.

13. The formulation as in claim 12 wherein the loop diuretic is furosemide.

14. The formulation as in claim 9 wherein the second pharmaceutically active compound is a potassium-sparing diuretic.

15. The formulation as in claim 14 wherein the potassium-sparing diuretic is amiloride.

16. The formulation as in claim 9 wherein the second pharmaceutical compound is a vasodilator.

17. The formulation as in claim 16 wherein the vasodilator is minoxidil.

18. The formulation as in claim 9 wherein the second pharmaceutically active compound is a calcium channel blocker.

19. The formulation according to claim 18 wherein the calcium channel blocker is selected from amlodipine, isradipine, felodipine, nicardipine, and nifedipine.

20. The formulation as in claim 9 wherein the second pharmaceutically active compound is an β-adrenoceptor blocker.

21. The formulation as in claim 20 wherein the β-adrenoceptor blocker is selected from atenolol, betaxolol, bisoprolol, carteolol, and propranolol.

22. The formulation as in claim 9 wherein the second pharmaceutically active compound is a renin inhibitor.

23. The formulation as in claim 22 wherein the renin inhibitor is enalkinen.

24. The formulation as in claim 9 wherein the second pharmaceutically active compound is an angiotensin converting enzyme inhibitor.

25. The formulation according to claim 24 wherein the angiotensin converting enzyme inhibitor is captopril or enalapril.

26. A method for blocking angiotensin II receptors in a subject, said method comprising administrating to a subject in need thereof an effective amount of the formulation according to claim 1.

27. A method for treating hypertension in a subject, said method comprising administrating to a subject in need thereof an effective amount of the formulation according to claim 1.

28. A method for treating congestive heart failure in a subject, said method comprising administrating to a subject in need thereof an effective amount of the formulation according to claim 1.

29. A method for treating renal failure in a subject, said method comprising administrating to a subject in need thereof an effective amount of the formulation according to claim 1.

30. A method for blocking angiotensin II receptors in a subject, said method comprising administrating to a subject in need thereof an effective amount of the formulation according to claim 9.

31. A method for treating hypertension in a subject, said method comprising administrating to a subject in need thereof an effective amount of the formulation according to claim 9.

32. A method for treating congestive heart failure in a subject, said method comprising administrating to a subject in need thereof an effective amount of the formulation according to claim 9.

33. A method for treating renal failure in a subject, said method comprising administrating to a subject in need thereof an effective amount of the formulation according to claim 9.

34. A process for the preparation of the formulation according to claim 1 comprising the steps of:
mixing eprosartan or a salt, solvate or hydrate thereof with a solubilizer and an emulsifier;
optionally adding one or more pharmaceutical acceptable excipients; and
filling the resulting liquid into a capsule.

35. A process for the preparation of the formulation according to claim 9 comprising the steps of:
mixing eprosartan or a salt, solvate or hydrate thereof with a solubilizer and an emulsifier;
adding and mixing a second pharmaceutical active compound until homogeneous;
optionally adding one or more pharmaceutical acceptable excipients; and filling the resulting liquid into a capsule.
